(19) United States
(12) Patent Application Publication
(10) Pub. No.: US 2017/0182284 A1
(43) Pub. Date: Jun. 29, 2017

(54) Device and Method for Generating Sound Signal

(71) Applicant: Yamaha Corporation, Hamamatsu-shi (JP)

(72) Inventors: Yuki UEYA, Iwata-shi (JP); Kiyoshi YAMAKI, Iwata-shi (JP); Morito MORISHIMA, Fukuroi-shi (JP)

(21) Appl. No.: 15/197,900

(22) Filed: Jun. 30, 2016

(30) Foreign Application Priority Data
Dec. 24, 2015 (JP) 2015-251445

(51) Int. Cl. A61M 21/02; G06F 3/16

(52) U.S. Cl. CPC A61M 21/02; G06F 3/165; G10H 2210/125; G10H 2210/391; G10H 2210/131; A61M 2021/0027

(57) ABSTRACT

A sound signal generation device includes a sound information acquirer acquiring biological information of a subject user, a change timing determiner determining a change timing that allows a first piece of sound information to be changed to a second piece of sound information in a cycle corresponding to the biological information acquired by the biological information acquirer, a sound signal generator generating a sound signal based on the second piece of sound information at a timing determined by the change timing determiner, and an amplitude of a waveform of a sound signal generated by the sound signal generator based on at least one piece of sound information, among a plurality of pieces of sound information including the first piece of sound information and the second piece of sound information, generally decreases from a maximum amplitude point, in which the amplitude is maximized, towards the end of the waveform, or generally increases from the start of the waveform towards a maximum amplitude point, in which the amplitude is maximized.

### STATEMENT REGARDING PRIOR DISCLOSURES BY THE INVENTOR OR A JOINT INVENTOR

YAMAKI, Kiyoshi, Yuki UEYA, and Morito MORISHIMA. “Suimin Wo Sasou Kankyo Ongaku” non-official translation (Sleep-inducing Environmental Music). The 40th Regular Scholarly Conference of the Japanese Society of Sleep Research. Utsunomiya Tobu Hotel Grande, Tochigi-ken, Japan. 2 Jul. 2015. Lecture.

YAMAKI, Kiyoshi, Yuki UEYA, Atsushi ISHIHARA, Morito MORISHIMA, Tomohiro HARADA, Keiki TAKADAMA, and Hiroshi KADOTANI. “Seitai Rizumu Ni Rendoushita Oto To Neiro No Chigai Ga Suimin Ni Oyobosu Eikyo” non-official translation (How Sounds and Tones Linked to Biological Rhythms Affect Sleep). The 40th Regular Scholarly Conference of the Japanese Society of Sleep Research. Utsunomiya Tobu Hotel Grande, Tochigi-ken, Japan. 3 Jul. 2015. Poster session.

UEYA, Yuki, Kiyoshi YAMAKI, Morito MORISHIMA. “Effects on Sleep by Sound and Tone Adjusted to Heartbeat and Respiration.” Medical Science Digest 25 Oct. 2015: 30-33. Print.

### BACKGROUND OF THE INVENTION

1. Field of the Invention

The present invention relates to device and a method for generating sound signals.

2. Description of the Related Art

In recent years, there has been proposed a technology for improving sleep and imparting relaxation by detecting biological information such as body motion, breathing and heartbeat, and generating a sound in accordance with the biological information (refer to, for example, Japanese Patent Application Laid-Open Publication No. Hei 04-269972). Also, there has been proposed a technology for adjusting at least one of a type, a volume, or a tempo of a sound generated in accordance with how relaxed a listener is (for example, refer to Japanese Patent Application Laid-Open Publication No. 2004-344284).

It has been noted that when generating a sound to improve a quality of sleep in a person listening to the sound (hereinafter, the subject user), if the sound is monotonous then sleep tends to be hindered by boredom or annoyance arising in the subject user listening to the sound.

The present invention has been made in view of these circumstances, and an object of the invention is to provide a technology that enhances a quality, etc., of sleep of the subject user.

### SUMMARY OF THE INVENTION

To achieve the abovementioned object, according to one aspect of the present invention, a sound signal generation device of the present invention includes: a biological information acquirer configured to acquire biological information of a subject user; a change timing determiner configured to determine a change timing that allows a first piece of sound information to be changed to a second piece of sound information in a cycle corresponding to the biological information acquired by the biological information acquirer; and a sound signal generator configured to generate a sound signal based on the second piece of sound information at a timing determined by the change timing determiner. An amplitude of a waveform of a sound signal generated by the sound signal generator based on at least one piece of sound information, among a plurality of pieces of sound information including the first piece of sound information and the second piece of sound information, generally decreases from a maximum amplitude point, in which the amplitude is maximized, towards the end of the waveform, or alternatively, generally increases from the start of the waveform towards the maximum amplitude point.

Rather than repeatedly generating the same sound signal based on the same sound information, in the present aspect, sound signals are generated by changing from the first piece of sound information to the second piece of sound information in a cycle based on acquired biological information, so that variations in sound signals can be increased. Thus, in the present aspect, the subject user's sleep is enhanced by enabling a cycle to be changed from the first piece of sound information to the second piece of sound information dependent on a cycle based on biological information acquired from the subject user. The cycle based on the acquired biological information does not necessarily have to coincide with a breathing cycle or a heartbeat cycle of the subject user and may be a cycle based on either a particular breathing cycle or heartbeat cycle constituting the acquired biological information. Thus, “the first sound information” is sound information before a change is made and “the second sound information” is the sound information when a change is made from the first sound information. The first and second pieces of sound information may be either the same or different.

If only a slight variation exists in an amplitude of the sound signals in the first piece of sound information and in an amplitude for the second piece of sound information, the subject user would not be able to distinctly perceive a cycle based on biological information, even when the first sound information is changed to the second sound information in a cycle based on the acquired biological information. In contrast, in the abovementioned aspect, the subject user is able to more readily perceive a cycle based on the acquired biological information since the waveform of the sound signals generated by the sound signal generator based on at least one piece of sound information, among a plurality of pieces of sound information, generally decreases from a maximum amplitude point, in which the amplitude is maximized, towards the end of the waveform, or alternatively, generally increases from the start of the waveform towards the maximum amplitude point. Accordingly, sleep can be more readily induced in the subject user within a shortened time period. As described above, it is possible to increase a variation in sound signals, and at the same time induce sleep in the subject user within a shortened time period.

The sound signal generation device according to the abovementioned aspect may be understood as a sound signal generation method. The sound signal generation method may be carried out by utilizing a computer-readable, non-transitory recording medium with a program stored therein, the program causing a computer to run the various processes of the sound signal generation method. The aforementioned effects of the invention are obtained by the sound signal generation method and also by the program stored in the recording medium.

According to another aspect of the present invention, the sound signal generation device of the present invention includes: a biological information acquirer configured to acquire biological information of a subject user; a repeat timing determiner configured to determine a repeat timing that allows a piece of sound information to be repeatedly generated in a cycle corresponding to the biological information acquired by the biological information acquirer; and a sound signal generator configured to generate a sound signal based on the piece of sound information at a timing determined by the repeat timing determiner. An amplitude of a waveform of a sound signal generated by the sound signal generator based on the piece of sound information generally decreases from a maximum amplitude point, in which the amplitude is maximized, towards the end of the waveform, or alternatively, generally increases from the start of the waveform towards the maximum amplitude point.

In this aspect the subject user is able to more readily perceive the cycle based on the acquired biological information, even when the same piece of sound information is played repeatedly since the waveform of the sound signals generated by the sound signal generator based on the piece of sound information generally decreases from a maximum amplitude point, in which the amplitude is maximized, towards the end of the waveform, or alternatively, generally increases from the start of the waveform towards the maximum amplitude point. Thus, sleep can more readily be induced in the subject user within a shortened time period.

The sound signal generation device according to this another aspect may be understood as a sound signal generation method. The sound signal generation method may be carried out by utilizing a computer-readable, non-transitory recording medium with a program stored therein, the program causing a computer to run the various processes of the sound signal generation method. The same effects of the invention as those of the sound signal generation device of this another aspect can be obtained by the sound signal generation method or by utilizing the program stored in the recording medium.

### BRIEF DESCRIPTION OF THE DRAWINGS

FIG. 1 is a diagram showing the overall configuration of the system including a sound signal generation device according to a first embodiment.

FIG. 2 is a block diagram showing a functional configuration of the sound signal generation device.

FIG. 3 is a block diagram showing an example configuration of a sound signal generator.

FIG. 4 is a diagram showing examples of sound information stored in a storage unit.

FIG. 5 is a waveform chart showing an example of sound information for a breathing-based cycle.

FIG. 6 is a waveform chart showing another example of sound information for a breathing-based cycle.

FIG. 7 is a flowchart showing an operation of the sound signal generation device.

FIG. 8 is a table explaining features of sound information used in sleep experiments.

FIG. 9 is a diagram showing an example waveform of sound information used in sleep experiments.

FIG. 10 is a graph showing the results of sleep experiments for all subject users.

FIG. 11 is a graph showing the results of sleep experiments for subject users belonging to a group having difficulty falling asleep.

FIG. 12 is a waveform chart showing an example of sound information for a breathing-based cycle.

FIG. 13 is a waveform chart showing another example of sound information for a breathing-based cycle.

FIG. 14 is a perspective view showing a configuration of a rocking bed according to a modification.

FIG. 15 is a block diagram showing a functional configuration of a sound signal generation device in a third embodiment.

FIG. 16 is a diagram showing examples of sound information stored in the storage unit.

FIG. 17 is a flowchart showing an operation of the sound signal generation device.

### DESCRIPTION OF THE EMBODIMENTS

In the following, an embodiment of the present invention will be described in detail with reference to the drawings.

### 1. First Embodiment

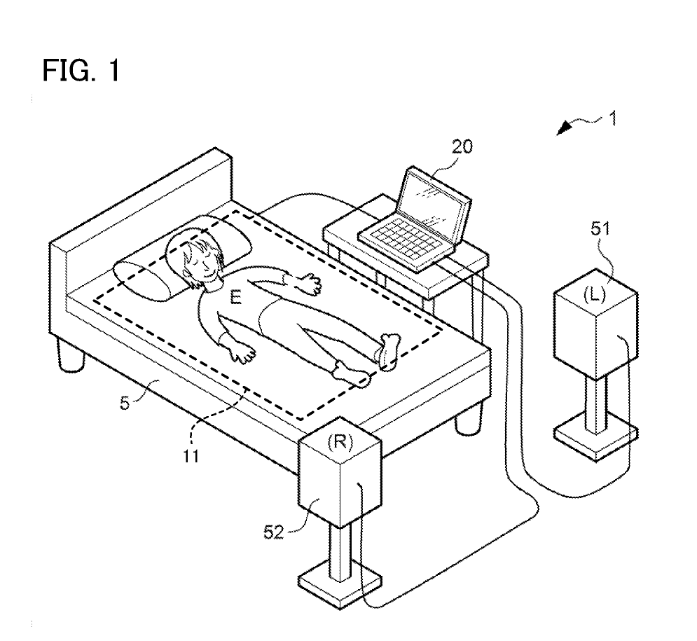

FIG. 1 is a diagram showing the overall configuration of a system 1, including a sound signal generation device 20 according to a first embodiment. As shown in the figure, the system 1 includes a sensor 11, a sound signal generation device 20 and speakers 51 and 52. The system 1 is directed to an improvement in aiding onset of sleep by enabling a subject user E lying on his/her back on a bed 5 to listen to a sound output from the speakers 51 and 52.

The sensor 11 has sheet-form piezoelectric elements and is disposed underneath a mattress on the bed 5. The sensor 11 detects the biological information of the subject user E when the subject user E lies down on the bed 5. The sensor 11 detects body motion originating from biological activities including the breathing and heartbeat of the subject user E. The detected signals including overlapping components of these biological activities are output from the sensor 11. For the sake of convenience, the figure shows a configuration in which the detected signals are transmitted by wire to the sound signal generation device 20, but the detected signals may instead be transmitted wirelessly.

The sound signal generation device 20 may acquire a breathing cycle BRm, a heartbeat cycle HRm and the body motion of the subject user E based on the detected signals (biological information) output from the sensor 11. Furthermore, the sound signal generation device 20 may estimate, based on the biological information output from the sensor 11, the physical and mental state of the subject user E and store information that relates to the sound output from the speakers 51 and 52, in association with an estimated physical and mental state (refer to the history table set out below). The sound signal generation device 20 may be, for example, a mobile terminal or a personal computer.

The speakers 51 and 52 are arranged in positions that allow the subject user E lying on his/her back to listen to stereo sound. Of the two, the speaker 51 is fitted with a built-in amplifier that amplifies the left (L) stereo sound signal output from the sound signal generation device 20 in emitting a sound. Similarly, the speaker 52 is fitted with a built-in amplifier that amplifies the right (R) stereo sound signal output from the sound signal generation device 20 in emitting a sound. It is of note that while in the present embodiment a configuration using the speakers 51 and 52 is employed, a configuration that enables the subject user E to listen to a sound through headphones may be also used.

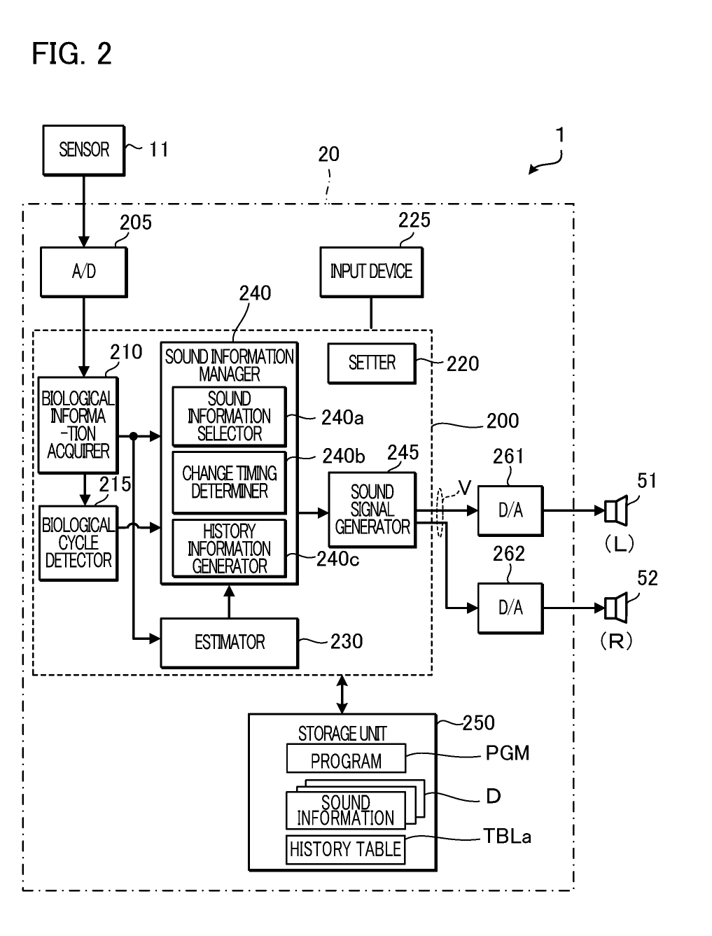

FIG. 2 is a diagram that shows a configuration of functional blocks of the sound signal generation device 20 of the system 1. As shown in this figure, the sound signal generation device 20 has an A/D converter 205, a controller 200, a storage unit 250, an input device 225, and D/A converters 261 and 262. The storage unit 250 is, for example, a non-transitory recording medium, and may be an optical recording medium (optical disc) such as a CD-ROM, or alternatively, any publicly known recording medium such as a magnetic recording medium or a semiconductor recording medium. A “non-transitory” recording medium referred to in the description of the present invention includes all types of recording media that may be read by a computer, except for a transitory, propagating signal, and volatile recording media are not excluded. The storage unit 250 stores a program PGM executed by the controller 200 and the various types of data used by the controller 200. For example, plural pieces of sound information (sound content) D and a history table TBLa are stored in the storage unit 250, the table TBLa storing an estimated physical and mental state of the subject user E in association with information on the sound output from the speakers 51 and 52. The program PGM may be provided in a form distributed through a communication network (not illustrated), which is installed in the storage unit 250.

The input device 225 is, for example, a touch screen, and is an input-output device having a display (for example, a liquid crystal screen) that shows various images under control of the controller 200, and an input unit into which a user (for example, the subject user E) inputs instructions for the sound signal generation device 20. The display and the input unit are constructed to be integral. The input device 225 may alternatively be configured as a device that is separate from the display, and that has plural operation units.

The controller 200 may, for example, include a processing device such as a CPU. By executing the program PGM stored in the storage unit 250, the controller 200 functions as a biological information acquirer 210, a biological cycle detector 215, a sound information manager 240, a setter 220, an estimator 230 and a sound signal generator 245. All or a part of these functions may be embodied in exclusive electronic circuitry. For example, the sound signal generator 245 may be configured using LSI (Large Scale Integration). The plural pieces of sound information D stored in the storage unit 250 may consist of any kind of data as long as they can generate sound signals V (V_{L} and V_{R}) in the sound signal generator 40. Examples of the sound information D include performance data indicating performance information such as notation and pitch, parameter data indicating parameters such as those controlling the sound signal generator 40, and waveform data.

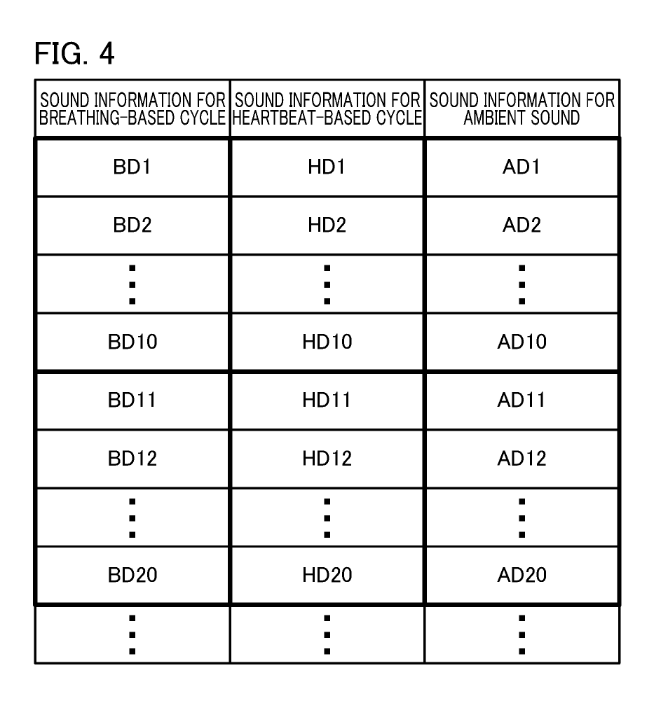

FIG. 4 shows an example of a plurality of pieces of sound information D stored in the storage unit 250. The same figure shows that the storage unit 250 stores sound information BD (BD1, BD2 . . . ) for a breathing-based cycle, sound information HD (HD1, HD2 . . . ) for a heartbeat-based cycle, and sound information AD (AD1, AD2 . . . ) for an ambient sound. As will be described later in more detail, the sound information BD for a breathing-based cycle is sound information that causes a sound signal to be generated in a cycle based on a breathing cycle BRm, the sound information HD for a heartbeat-based cycle is sound information that causes a sound signal to be generated in a cycle based on a heartbeat cycle HRm, and the sound information AD for an ambient sound is sound information that causes a sound signal to be generated in a cycle related to neither the breathing cycle BRm nor the heartbeat cycle HRm.

The A/D converter 205 converts the signals detected by the sensor 11 into digital signals. The biological information acquirer 210 acquires and temporarily stores the converted digital signals in the storage unit 250. The biological cycle detector 215 detects the biological cycles of the subject user E based on the biological information stored in the storage unit 250. According to the present embodiment, the biological cycle detector 215 detects the heartbeat cycle HRm and the breathing cycle BRm as the biological cycles, and supplies the detected cycles to the sound information manager 240. The estimator 230 estimates the physical and mental state of the subject user E based on the acquired biological information stored in the storage unit 250, to supply the information indicating the estimated physical and mental state to the sound information manager 240.

The setter 220 makes various settings. The sound signal generation device 20 may generate multiple sound signals V and cause the speakers 51 and 52 to emit multiple kinds of the sound signals V so as to prevent boredom in the subject user E. The setter 220 sets the tone, etc., of a sound according to an input made by the subject user E into the input device 225, and temporarily stores the details of the setting in the storage unit 250 as setting data.

According to the present embodiment, the estimator 230 estimates a physical and mental state (e.g., stage of sleep) of the subject user E based on the detection results of the sensor 11, from the time the subject user E rests to the time he/she falls asleep, and to the time he/she wakes up. The estimator 230 estimates which of the following stages the subject user E is in: for example, “awake”, “light sleep”, “deep sleep”, or “REM sleep”. It is of note that “deep sleep” as well as “light sleep” may be “non-REM sleep”. Generally speaking, a person's breathing cycle BRm and heartbeat cycle HRm tend to elongate during a period when he/she falls from wakefulness into deep sleep. There is also a tendency for these cycles to vary less during such a period. In addition, the deeper the sleep, the less body motion there is. In view of the above, the estimator 230 combines the change in the breathing cycle BRm, the change in the heartbeat cycle HRm and the number of times the body moves in one unit time and compares the combined results with plural thresholds to estimate a physical and mental state based on the detected signals of the sensor 11.

The sound information manager 240 is a functional element that executes various functions relating to the processing of the sound information D. Specifically, the sound information manager 240 has a sound information selector 240a, change timing determiner 240b and history information generator 240c as shown in FIG. 2. The sound information selector 240a selects which piece of sound information D to read, among plural pieces of sound information D stored in the storage unit 250, based on the setting data stored in the storage unit 250. Then the sound information selector 240a supplies designation data that designates the selected sound information D to the sound signal generator 245. Specifically, the sound information selector 240a selects at least one of the following based on the setting data stored in the storage unit 250: the sound information BD for a breathing-based cycle; the sound information HD for a heartbeat-based cycle; and the sound information AD for an ambient sound. The history information generator 240c stores the identifier for the physical and mental state estimated by the estimator 230 in association with the identifier for the selected sound information with the time at which the processing was carried out (e.g., the time at which the sound signal based on the sound information D was generated) in a history table TBLa stored in the storage unit 250.

The change timing determiner 240b determines a timing at which to change from the first sound information D to the second sound information D. Specifically, the change timing determiner 240b determines the timing at which to change from the first sound information D to the second sound information D, such that the change is carried out in a cycle based on biological information acquired by the biological information acquirer 210; for example, a cycle obtained by multiplying by a predetermined number the breathing cycle BRm or the heartbeat cycle HRm acquired from the biological information. Here, the first sound information D is the sound information before the change is made, and the second sound information D is the sound information to which the change is made. That is, when the first sound information D is defined as the sound information D based on which the sound signal V has most recently been generated, the second sound information D is defined as the sound information D based on which the sound signal V is generated subsequent to the first sound information D as a result of the sound information selector 240a sequentially selecting the sound information D. In other words, the first sound information D and the second sound information may be any two pieces of sound information D, and the sound signal V generated based on the second sound information piece D follows the sound signal V generated based on the first sound information piece D.

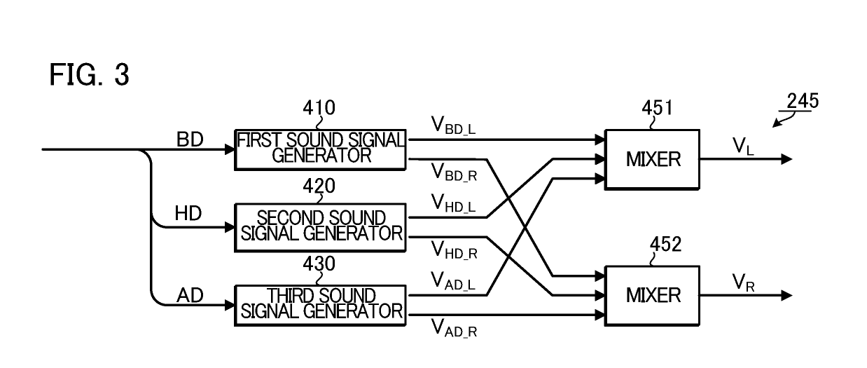

The sound signal generator 245 acquires, from the storage unit 250, the sound information D corresponding to the designation data supplied from the sound information selector 240a at a timing when the determination is made by the change timing determiner 240b, and then generates the sound signal V based on the acquired sound information. FIG. 3 shows a detailed configuration of the sound signal generator 245. The sound signal generator 245 has first, second, and third sound signal generators 410, 420 and 430 and mixers 451 and 452.

The first sound signal generator 410 generates a sound signal V_{BD}(V_{BD}_{_}_{L} and V_{BD}_{_}_{R}) linked to the breathing cycle BRm, based on the sound information BD for a breathing-based cycle, so that sound linked to breathing is obtained. The second sound signal generator 420 generates a sound signal V_{HD} (V_{HD}_{_}_{L} and V_{HD}_{_}_{R}) linked to the heartbeat cycle HRm, based on the sound information HD for a heartbeat-based cycle, so that sound linked to heartbeat is obtained. The third sound signal generator 430 generates, in a cycle not linked to either the breathing cycle BRm or the heartbeat cycle HRm, a sound signal V_{AD} (V_{AD}_{_}_{L} and V_{AD}_{_}_{R}) based on the sound information AD for an ambient sound.

Specifically, according to the present embodiment, the first, second, and third sound signal generators 410, 420 and 430 acquire, from the storage unit 250, second sound information D (corresponding to one of the sound information BD, HD or AD) selected by the sound information selector 240a individually for each of the sound information BD for a breathing-based cycle, the sound information HD for a heartbeat-based cycle, and the sound information AD for an ambient sound. The acquisition of the sound information D is performed at a timing respectively determined by the change timing determiner 240b for each of the first, second, and third sound signal generators 410, 420 and 430. The first, second, and third sound signal generators 410, 420 and 430 each generate the sound signal V (V_{BD}, V_{HD} or V_{AD}) based on respective acquired second sound information D and emit the sound signals V_{BD} (V_{BD}_{_}_{L} or V_{BD}_{_}_{R}), V_{HD} (V_{HD}_{_}_{L} or V_{HD}_{_}_{R}) or V_{AD} (V_{AD}_{_}_{L} and V_{AD}_{_}_{R}) in a stereo, two-channel digital format.

The mixer 451 combines (adds) the left (L) sound signals of V_{BD}_{_}_{L}, V_{HD}_{_}_{L} and V_{AD}_{_}_{L} that are individually output from a respective one of the first, second and third sound signal generators 410, 420 and 430, and generates the sound signal V_{L} that is to be output. Similarly, the mixer 452 generates the sound signal V_{R} that is to be output, by combining the right (R) sound signals of V_{BD}_{_}_{R}, V_{HD}_{_}_{R} and V_{AD}_{_}_{R} that are individually output from the respective one of the sound signal generators 410, 420 and 430. The ratio of the mixture is controlled by control signals output from the sound information manager 240. The D/A converter 261 converts the left (L) sound signal V_{L} that has been combined by the mixer 451 into an analog signal for output. Similarly, the D/A converter 262 converts the right (R) sound signal V_{R} that has been combined by the mixer 452 into an analog signal for output.

In this embodiment, the change timing determiner 240b determines the timing at which to change from the first sound information D to the second sound information D so that the change is made in a cycle based on biological information of the subject user E. The first, second and third sound signal generators 410, 420 and 430 each then generate a sound signal based on the first sound information D at a timing determined by the change timing determiner 240b (i.e., a change is made from the first sound information D to the second sound information D). The abovementioned process is defined as “generating sound information D (the second sound information D that is the sound information after the change is made) in a manner linked to the biological cycles” or “changing from the first sound information D to the second sound information D in a manner linked to the biological cycles”.

The duration of playing the sound information BD for a breathing-based cycle, stored in the storage unit 250, is set longer than the average breathing cycle BRm of a person. The playing duration is set in this way because the sound information BD for a breathing-based cycle is intended to change to a new sound information BD according to a cycle based on the breathing cycle BRm, and it is preferable for the sound information BD to be played from the beginning to the end of one breathing cycle BRm. The same can be said for the sound information HD for a heartbeat-based cycle, and accordingly the duration of playing the sound information HD for a cycle based on heartbeat is set longer than the average heartbeat cycle HRm of a person.

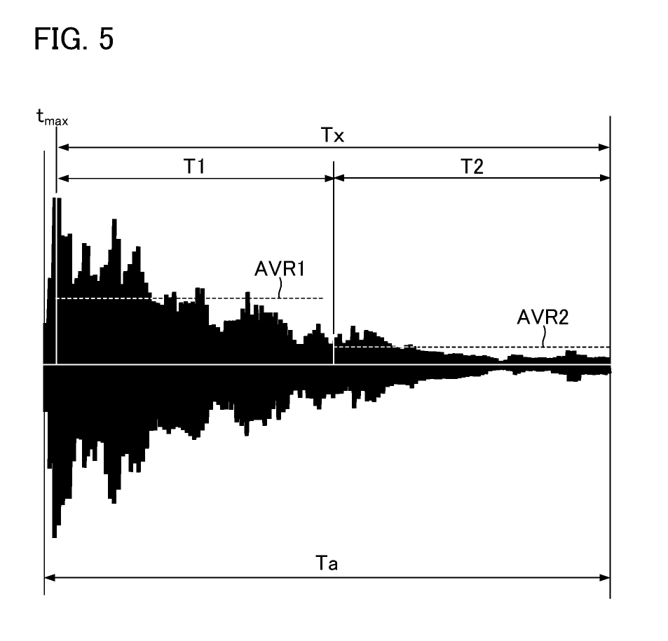

FIG. 5 shows an example of a waveform of the sound signal V generated by the sound signal generator 245 based on the sound information BD for a breathing-based cycle. As the figure shows, the full playing duration Ta of the waveform of the sound signal V corresponding to the sound information BD for a breathing-based cycle is, for example, 10 seconds. The amplitude of the waveform of FIG. 5 generally decreases from the beginning towards the end. The expression “generally decreases” means that although when viewing a waveform over a short time span both increases and decreases in amplitude may be present, when the waveform is viewed as a whole a tendency towards a general decrease in amplitude is present. In the following explanation, such a waveform will be referred to as a decreasing type of waveform. Regarding the waveform of the sound signal V corresponding to the sound information BD for a breathing-based cycle, the mean amplitude AVR2 of the waveform in the second period T2 is smaller than the mean amplitude AVR1 of the waveform in the first period T1, when the period Tx, which is the period between the maximum amplitude point tₘₐₓ and the end of the waveform, is divided in half, i.e. into the first period T1 and the second period T2. Here, the maximum amplitude point tₘₐₓ indicates the time at which the amplitude maximizes in a waveform of the sound information BD for a breathing-based cycle.

In the present embodiment, the sound signal generator 245 generates a sound signal based on the second sound information D so that a change is made from the first sound information D to the second sound information D in a cycle based on the acquired biological information of the subject user E. Compared to loop-playback in which the same sound information D is repeatedly played, the sound signal generation device 20 of the present embodiment provides an advantage in that it helps prevent boredom from occurring in the subject user E. Moreover, the sound signal generation device 20 of the present embodiment is expected to lead the subject user E into a relaxed state, which is another advantage, since the subject user E is able to perceive his/her biological cycles upon a change from the first sound information D to the second sound information D in a cycle based on his/her biological information. Even in case of loop-playback in which the same sound information D is played repeatedly, the subject user E can perceive his/her biological cycles by having a piece of sound information D with a decreasing type waveform, such as the one shown in FIG. 5, played repeatedly. Accordingly, as long as the sound information D has a decreasing type waveform, the same effects as those for when a piece of first sound information D and a piece of second sound information D differ can be achieved even if the piece of first sound information D and the piece of second sound information D are the same. The same can be said for the second embodiment which will be described later. Here, a cycle based on acquired biological information indicates the cycle corresponding to the biological cycle (breathing cycle BRm or heartbeat cycle HRm) detected from the biological information acquired by the biological information acquirer 210. In this regard, a cycle based on the acquired biological information may be referred to as a cycle based on biological cycles (biological rhythms).

Generally speaking, once a person falls asleep, his/her biological cycles such as heartbeat and breathing cycles slow down compared to when he/she is awake. From this, it is expected that, by having the subject user E listen to a sound in a cycle based on his/her acquired biological information (for example, in a cycle 5% longer than the biological cycle), the time from when the subject user E goes to bed to when he/she falls asleep will shorten.

The reason why a decreasing type waveform shown in FIG. 5 was chosen is as follows. As the figure shows, in the decreasing type waveform, the mean amplitude is smaller in the second period T2 than in the first period T1, and therefore, the amplitude of the waveform decreases within a single cycle Ta. If the amplitude of the waveform is constant or lacking in variation, the subject user E may not readily be able to perceive the cycle based on his/her acquired biological information, even when a change is made from the first sound information D to the second sound information D in a cycle based on the acquired biological information. In contrast, in the decreasing type waveform, the amplitude of the waveform generally decreases from the maximum amplitude point tₘₐₓ towards the end of the waveform, allowing the volume of the sound listened to by the subject user E to change in a cycle based on his/her acquired biological information. Therefore, by employing a decreasing type of sound information D, the subject user E can more distinctly perceive the cycle based on his/her acquired biological information, thus inducing sleep in him/her more quickly after going to bed.

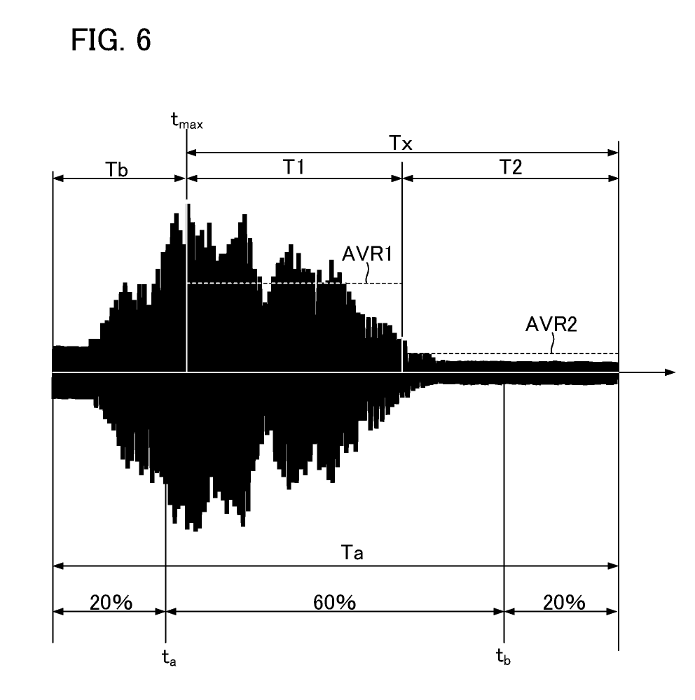

Next, FIG. 6 shows another example waveform of the sound signal V generated by the sound signal generator 245 based on the sound information BD for a breathing-based cycle. The amplitude of the waveform in the figure first gradually increases and then generally decreases after it is maximized. Such a waveform will be referred to as a decreasing-after-increasing type waveform. At the beginning of the decreasing-after-increasing type waveform, there is a period Tb in which the amplitude increases. Regarding also the waveform of the decreasing-after-increasing type, the mean amplitude AVR2 of the waveform in the second period T2 is smaller than the mean amplitude AVR1 of the waveform in the first period T1, when the period Tx, which is the period between the maximum amplitude point tₘₐₓ and the end of the waveform, is divided in half, i.e. into the first period T1 and the second period T2.

As in the decreasing type waveform, in the decreasing-after-increasing type waveform, the amplitude of the waveform generally decreases from the maximum amplitude point tₘₐₓ towards the end of the waveform, thus allowing the volume of the sound listened to by the subject user E to change in a cycle based on his/her acquired biological information. Therefore, by selecting the sound information D based on which the sound signal V having the decreasing-after-increasing type waveform is generated, the subject user E can more distinctly perceive the cycle based on his/her acquired biological information, thus inducing sleep in him/her more quickly after going to bed. Here, in the decreasing type waveform of FIG. 5 and the decreasing-after-increasing type waveform of FIG. 6, it is preferable that the mean amplitude AVR2 be equal to or less than 70% of the mean amplitude AVR1. When AVR2 is equal to or less than 70% of AVR1 the subject user E can more readily perceive his/her own biological cycles. In the examples shown in FIGS. 5 and 6, the time period Tx between the maximum amplitude point tₘₐₓ and the end of the waveform is divided in half. However, it is also possible to divide the period Tx by three or four. In such a case, the mean amplitude of each period generally decreases towards the end of the waveform.

Furthermore, in a waveform of the sound signal V corresponding to the sound information D of the decreasing-after-increasing type, when the entire time from the start to the end of the waveform is deemed 100%, it is preferable that the maximum amplitude point tₘₐₓ comes within the range between a time point tₐ, at which 20% of time has passed from the start of the waveform, and a time point t_{b}, at which 20% of time remains until the end of the waveform. If the maximum amplitude point tₘₐₓ is within this range, the subject user E can more distinctly perceive the process in which the amplitude increases to its maximum point and the process in which the amplitude decreases from the maximum point. Accordingly, it is expected that the subject user also will more readily perceive the volume change in the cycle based on his/her acquired biological information and that sleep will be induced in the user. It is noted that, preferably, all pieces of sound information D stored in the storage unit 250 should be of either the aforementioned decreasing type or decreasing-after-increasing type. However, not all pieces of sound information D stored in the storage unit 250 and selected by the sound information selector 240a are required to be of the abovementioned decreasing type or decreasing-after-increasing type, and only at least one of the pieces of sound information D stored in the storage unit 250 is required to be of such a type. In other words, it is sufficient for the waveform of the sound signal V that corresponds to at least one among the pieces of sound information D selected by the sound information selector 240a to have its amplitude generally decrease from the maximum amplitude point tₘₐₓ, at which the amplitude is maximized, towards the end of the waveform.

Returning to FIG. 4, a plurality of pieces of the sound information BD for a breathing-based cycle is managed in groups. In this example, the first group contains pieces of sound information BD1 through BD10 for a breathing-based cycle, and the second group contains pieces of sound information BD11 through BD20 for a breathing-based cycle. Pieces of sound information BD of the decreasing type belong to the first group. For example, the first group includes pieces of sound information BD that represent the sound of bells. Pieces of sound information BD of the decreasing-after-increasing type belong to the second group. Alternatively, the grouping may be made according to musical instruments, such as a harp or guitar. It is noted that each of the plural pieces of sound information BD for a breathing-based cycle that belong to the different groups differs from one another. The full playing duration Ta of a waveform of each sound signal V of the sound information BD for a breathing-based cycle is, for example, 10 seconds.

The playing duration of each sound information HD for a heartbeat-based cycle is 1.2 seconds. As with the sound information BD for a breathing-based cycle, the sound information HD for a heartbeat-based cycle is also managed in groups. In this example, the first group contains pieces of sound information HD1 through HD10 for a heartbeat-based cycle, and the second group contains pieces of sound information HD11 through HD20 for a heartbeat-based cycle. The first group includes pieces of sound information HD corresponding to the sound signal V that has a waveform of the decreasing type, formed by a sound of drums, for example. The second group includes pieces of sound information HD of the decreasing type representing a sound of wind chimes, for example. It is noted that each of the plural pieces of sound information HD for a heartbeat-based cycle that belong to the different groups differs from one another.

The playing duration of the sound information AD for an ambient sound is 100 seconds. As with the sound information BD for a breathing-based cycle, the sound information AD for an ambient sound is also managed in groups. In this example, the first group contains pieces of sound information AD1 through AD10 for an ambient sound, and the second group contains pieces of sound information AD11 through AD20 for an ambient sound. The first group includes pieces of sound information AD that represent the sound of waves. The second group includes pieces of sound information AD that represent the sound of a creek. Alternatively, the groups can be of pieces of sound information AD representing the sounds of wind, or those representing the sounds of crowded streets.

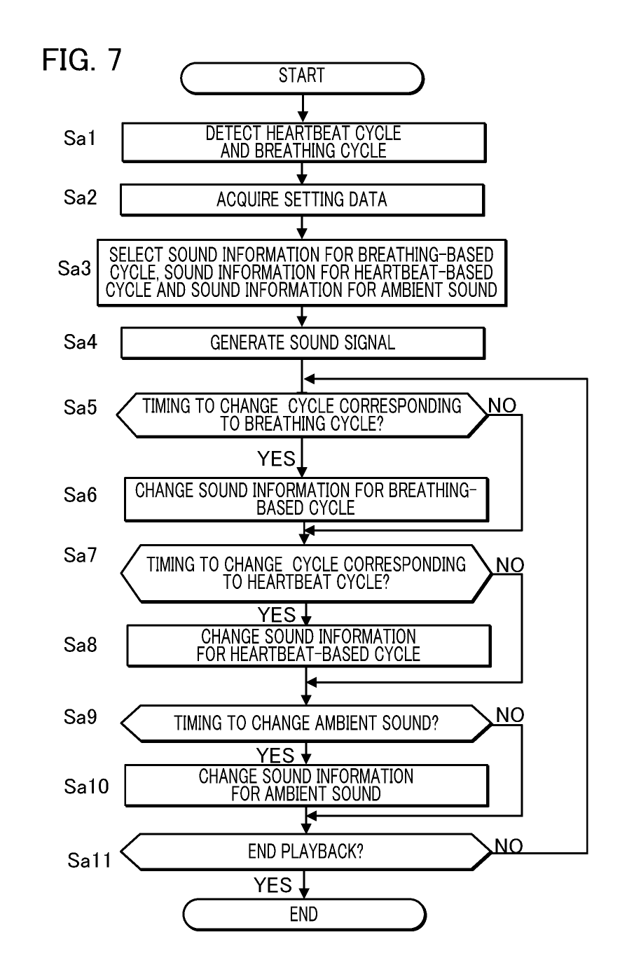

Next, operation of the system 1 will be described. FIG. 7 is a flowchart showing the operation of the sound signal generation device 20. First, the biological cycle detector 215 detects the heartbeat cycle HRm and the breathing cycle BRm of the subject user E based on the detection signals indicating the biological information of the subject user E acquired by the biological information acquirer 210 (Sa1). The frequency band of the breathing components imposed upon the detected signals generally is between 0.1 Hz and 0.25 Hz, inclusively or exclusively. The frequency band of the heartbeat components imposed upon the detected signals generally is between 0.9 Hz and 1.2 Hz, inclusively or exclusively. The biological cycle detector 215 extracts from the detected signals signal components of the frequency band corresponding to the breathing component and detects the breathing cycle BRm of the subject user E based on the extracted components. Furthermore, the biological cycle detector 215 extracts from the detected signals signal components of the frequency band corresponding to the heartbeat component and detects the heartbeat cycle HRm of the subject user E based on the extracted components. It is noted that the biological cycle detector 215 constantly detects the heartbeat cycle HRm and the breathing cycle BRm of the subject user E even during execution of each of the processes described below.

Upon acquiring from the storage unit 250 the setting data that has been set by the setter 220 (Sa2), the sound information selector 240a determines, based on the setting data, the group from which the sound information D is selected, with regard to each of the sound information BD for a breathing-based cycle, the sound information HD for a heartbeat-based cycle and the sound information AD for an ambient sound. Here, the setting data includes information that designates at least one of the following: the sound information BD for a breathing-based cycle; the sound information HD for a heartbeat-based cycle; or the sound information AD for an ambient sound. The setting data may also include information that indicates a desired tone or that indicates a kind of musical instrument being played selected by the subject user E.

Regarding this operation example it is assumed that all of the following are designated by the setting data: the sound information BD for a breathing-based cycle; the sound information HD for a heartbeat-based cycle; and the sound information AD for an ambient sound. However, a configuration in which the setting data designates at least one among the above is possible. For example, a configuration such as the following is possible; namely, a configuration in which the setting data designates the sound information BD for a breathing-based cycle and the sound information AD for an ambient sound but not the sound information HD for a heartbeat-based cycle, as a result of which the sound information selector 240a determines the group from which sound information shall be selected with regard to each of the sound information BD for a breathing-based cycle and the sound information AD for an ambient sound.

According to a prescribed rule (in this operation example, the rule is random selection), the sound information selector 240a selects any one of the plural pieces of sound information D belonging to the group determined as the source of selection of the sound information D. In a configuration in which the sound information D is selected randomly, it is possible for the same piece of sound information D for a breathing-based cycle to be selected repeatedly. Therefore, the first sound information D before the change is made and the second sound information D after the change has been made may be identical. When the first sound information D and the second sound information D are different, the variation of the sounds that the subject user E listens to may be increased.

Next, the sound information selector 240a selects, according to a prescribed rule, each of the following from the respective groups that have been determined: a piece of the sound information BD for a breathing-based cycle; a piece of the sound information HD for a heartbeat-based cycle; and a piece of the sound information AD for an ambient sound (Sa3). In this example, the rule is to make the selection randomly. In the present description, randomness is a notion including pseudo-randomness. For example, the selection of the sound information D from the respective groups may be made using pseudo-random signals generated in M series generators. The sound signal generator 245 then generates the sound signal V using the randomly selected pieces of sound information BD for a breathing-based cycle, sound information HD for a heartbeat-based cycle and sound information AD for an ambient sound (Sa4).

Subsequently, the change timing determiner 240b determines whether or not the current time is the change timing based on a cycle corresponding to the breathing cycle BRm of the subject user E (Sa5). More specifically, the change timing determiner 240b determines whether or not the current time is the time at which the amount of time corresponding to a cycle based on the breathing cycle BRm has elapsed since the time at which the sound information BD for a breathing-based cycle started to play (for example, the acquisition time of the sound information BD), the sound information BD here being the sound information most recently acquired from the storage unit 250 by the sound signal generator 245. Here, the cycle based on the breathing cycle BRm does not necessarily have to coincide with the detected breathing cycle BRm, and only has to be a cycle obtained under a particular relationship with the breathing cycle BRm. For example, the mean value of the breathing cycles BRm within a prescribed period may be calculated and then multiplied by K (K standing for a selected value fulfilling the equation 1≦K≦1.1), the breathing cycles BRm having been detected by the biological cycle detector 215. In this example, the change timing determiner 240b sets the change timing of the sound information BD for a breathing-based cycle by multiplying the mean value by 1.05. In this case, if the mean value of the breathing cycle BRm of the subject user E is 5 seconds, the change cycle would be 5.25 seconds. A person's breathing cycle BRm tends to be longer when he/she feels relaxed. Therefore, by setting the change cycle slightly longer than the measured breathing cycle BRm, it is expected that a person will feel relaxed and thus be able to fall asleep quickly.

When the determination conditions in step Sa5 are met, the change timing determiner 240b supplies to the sound signal generator 245 a timing signal that instructs the sound signal generator 245 to generate a sound signal based on new sound information BD for a breathing-based cycle (the second sound information BD). Once the timing signal has been supplied, the first sound signal generator 410 of the sound signal generator 245 acquires from the storage unit 250 the sound information BD for a breathing-based cycle selected by the sound information selector 240a as the second sound information BD. Then the first sound signal generator 410 generates the sound signal V_{BD} based on the acquired second sound information BD (Sa6). The selection of the sound information BD by the sound information selector 240a is performed upon each occurrence of the timing for generating a sound signal based on the second sound information BD for a breathing-based cycle (the timing for changing from the first sound information BD to the second sound information BD). The selected sound information BD is supplied to the sound signal generator 245 along with the timing signal.

When the determination conditions in step Sa5 are not met or when the processing of step Sa6 is completed, the change timing determiner 240b determines whether or not the change timing is based on the cycle corresponding to the heartbeat cycle HRm of the subject user E (Sa7). Here, the cycle based on the heartbeat cycle HRm does not necessarily have to coincide with the detected heartbeat cycle HRm, and only has to be a cycle obtained under a particular relationship with the heartbeat cycle HRm. For example, the mean value of the detected heartbeat cycle HRm within a prescribed period may be calculated and then multiplied by L (L standing for a selected value fulfilling the equation 1≦L≦1.1). In this example, the change timing determiner 240b sets the change timing of the sound information HD for a heartbeat-based cycle by multiplying the mean value by 1.02. In this case, if the mean value of the heartbeat cycle HRm of the subject user E is 1 second, the change cycle would be 1.02 seconds. A person's heartbeat cycle HRm tends to be longer when he/she feels relaxed. Therefore, by setting the change cycle slightly longer than the measured heartbeat cycle HRm, it is expected that the subject user E will feel relaxed and thus he/she will be able to fall asleep quickly.

When the determination conditions in step Sa7 are met, the change timing determiner 240b supplies to the sound signal generator 245 a timing signal that instructs the sound signal generator 245 to generate a sound signal based on new sound information HD for a heartbeat-based cycle (the second sound information HD). Once the timing signal is supplied, the second sound signal generator 420 of the sound signal generator 245 then acquires from the storage unit 250 the sound information HD for a heartbeat-based cycle that has been selected by the sound information selector 240a as the second sound information HD. Then the second sound signal generator 420 generates the sound signal V_{HD} based on the acquired second sound information HD (Sa8). The selection of the sound information HD by the sound information selector 240a is performed every time the timing to generate a sound signal for the new sound information HD for a heartbeat-based cycle (the timing to change from the first sound information HD to the second sound information HD) occurs. The selected second sound information HD is supplied to the sound signal generator 245 along with the timing signal.

Meanwhile, when the determination conditions in step Sa7 are not met or when the processing of step Sa8 is finished, the change timing determiner 240b determines whether or not it is the timing to change the sound information AD for an ambient sound (Sa9). The change cycle for the sound information AD for the ambient sound may be freely set. For example, the cycle could be 100 seconds, or the time at which the playback of a single piece of sound information AD for the ambient sound ends could be the change timing. Alternatively, the change timing could be set according to a cycle obtained by multiplying by Q (Q standing for a natural number equal to or larger than 2) the cycle corresponding to the breathing cycle BRm or the heartbeat cycle HRm. For example, when Q is 10, the sound information AD for an ambient sound would be changed in a cycle that is ten times the cycle corresponding to the change cycle of the sound information BD for a breathing-based cycle. In this case, the change timing for the sound information BD for a breathing-based cycle and the change timing for the sound information AD for an ambient sound may or may not coincide.

When the determination conditions in step Sa9 are met, the change timing determiner 240b supplies to the sound signal generator 245 a timing signal that instructs the sound signal generator 245 to generate a sound signal based on new sound information AD for an ambient sound (the second sound information AD). Once the timing signal is supplied, the third sound signal generator 430 of the sound signal generator 245 then acquires from the storage unit 250 the sound information AD for an ambient sound selected by the sound information selector 240a as the second sound information AD. Then the third sound signal generator 430 generates the sound signal V_{AD} based on the acquired second sound information AD (Sa10). The selection of the sound information AD by the sound information selector 240a is performed upon occurrence of each timing for generating a sound signal based on the second sound information AD for an ambient sound (the timing for changing from the first sound information AD to the second sound information AD). The selected sound information AD is supplied to the sound signal generator 245 along with the timing signal. As when selecting the sound information BD and the sound information HD, the sound information selector 240a selects the sound information AD in a random manner, thus increasing the variation in the sounds the subject user E listens to.

Meanwhile, when the determination conditions in step Sa9 are not met or when the processing of step Sa10 is completed, the controller 200 determines whether or not to end the playback of the sound information D (Sa11). When an input instruction instructing the end of playback is input via the input device 225 or when the playing duration that has been set in advance exceeds the current time (Sa11: Yes), the controller 200 ends the sound signal generation process of the present embodiment. On the other hand, when the determination conditions in step Sa11 are not met, the controller 200 returns the processing to step Sa5 and let the processes of steps Sa5 through Sa10 repeat themselves. The biological cycle detector 215 constantly detects the heartbeat cycle HRm and the breathing cycle BRm, so when there is a change in the heartbeat cycle HRm and the breathing cycle BRm, to follow the change, the change cycle for the sound information BD for a breathing-based cycle and the change cycle for the sound information HD for a heartbeat-based cycle also change. In some cases (i.e., in cases where the change cycle is set at Q times the heartbeat cycle HRm or the breathing cycle BRm), the change cycle for the sound information AD for an ambient sound also changes.

Accordingly, in the first embodiment, sounds with various different tones may be played even with a limited number of pieces of sound information. In particular, because the sound signal generation device 20 of the present invention randomly selects sound information D, rather than repeatedly selecting the same sound information D, it is possible to alleviate discomfort the listener may experience when, for example, the sound becomes monotonous or annoying to the listener. Furthermore, it is widely known that so-called relaxing or healing sounds that cause a (alpha) waves to more frequently occur in brain wave patterns have natural fluctuation components. Through random selection, it is possible for the plural pieces of sound information D to impart fluctuation effects to the sounds obtained in playing them. Moreover, by way of the subject user E's setting operation of the setter 220, combinations of sounds can be created in which each of the sound information is either played or not played, as follows: the sound information BD for a breathing-based cycle; the sound information HD for a heartbeat-based cycle; and the sound information AD for an ambient sound. In addition, since pieces of the sound information D stored in the storage unit 250 include a piece of sound information from which the waveform of the sound signal V is generated in the sound signal generator 245, either the decreasing type or the decreasing-after-increasing type, if one such type is selected, the volume of the sound to which the subject user E listens may be changed in a cycle based on his/her biological cycles. Thus in the present embodiment the subject user E is able to more distinctly perceive the cycle based on his/her acquired biological cycles as a result. Thus it is expected that the subject user E will fall asleep more quickly after going to bed.

### 2. Sleep Experiments

The inventors of the present invention, using the biological information (heartbeat cycle HRm and breathing cycle BRm) of a subject user acquired from a sensor, conducted experiments to induce sleep in the subject user by having him/her listen to first sound information D and second sound information D changing from one to the other in a change cycle slightly slower than the breathing cycle BRm.

### 2-1. Methodology of the Experiments

The sleep experiments were conducted in accommodation, with 22 people comprising the subject users, aged between 26 and 51, and having an average age of 43, including 2 women, the subject users being observed each night between 2200 h, which was the time they went to bed, and 0600 h, which was the time they got up the next morning. The subject users listened to the same type of sound during each night's stay. The system used in the present set of experiments included a sensor 11, a sound signal generation device 20 and speakers 51 and 52, each identical to those shown in FIG. 1. The sensor 11, which is in the form of a sheet, able to measure heartbeat, breathing and body motion in a non-invasive, non-restraining manner was used to acquire biological information. The sensor 11 was connected to the sound signal generation device 20. The sound signal generation device 20 controlled the timing at which the first sound information D is changed to the second sound information D according to the heartbeat cycle HRm and breathing cycle BRm detected from the acquired biological information. The sound signal generation device 20 then determined whether or not the subject users had fallen asleep based on body motion information that had been separated from the acquired biological information. The sleep latency time was deemed to be a time from when the subject users went to bed to when they fell asleep.

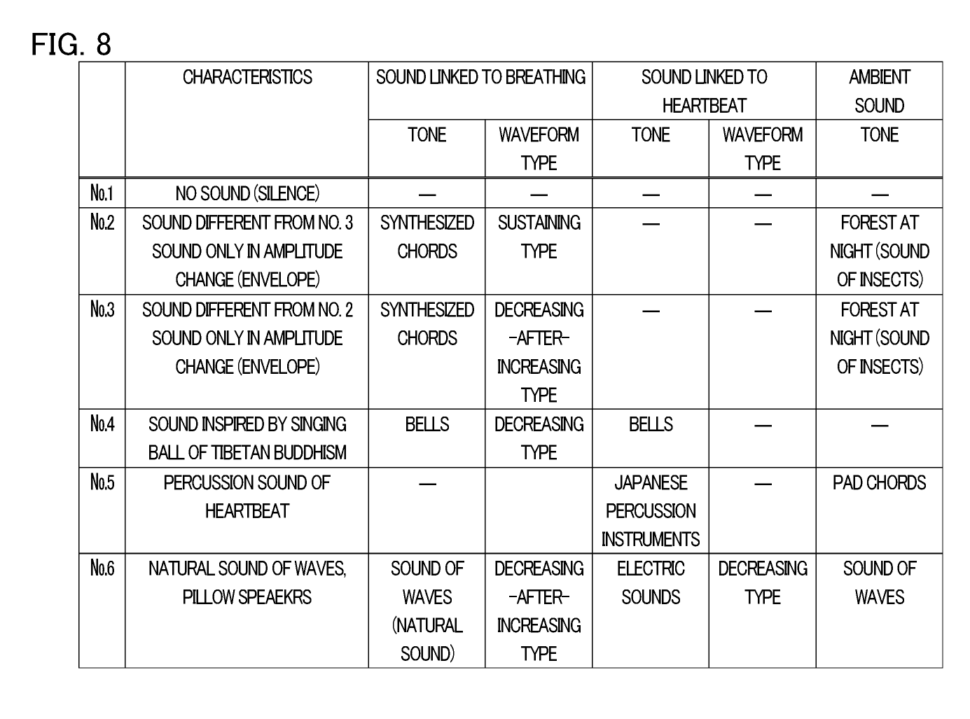

FIG. 8 shows the characteristics of the 6 kinds of sounds that were used in the experiments. The sound linked to breathing shown in FIG. 8 was obtained by causing the speakers 51 and 52 to emit the sound signal V based on the sound information BD for a breathing-based cycle. Specifically, plural sound signals V were generated by sequentially changing from the first sound information BD to the second sound information BD in a cycle based on the breathing cycle, and the sound linked to breathing was emitted. Similarly, by causing the speakers 51 and 52 to emit the sound signal V based on the sound information HD for a heartbeat-based cycle, the sound linked to heartbeat was obtained. Specifically, plural sound signals V were generated by sequentially changing from the first sound information HD to the second sound information HD in a cycle based on the heartbeat cycle, and the sound linked to heartbeat was emitted. The ambient sound was obtained by causing the speakers 51 and 52 to emit the sound signal V based on the sound information AD for an ambient sound. Specifically, plural sound signals V were generated by sequentially changing from the first sound information AD to the second sound information AD in a cycle based on neither the breathing cycle nor the heartbeat cycle, and the ambient sound was emitted.

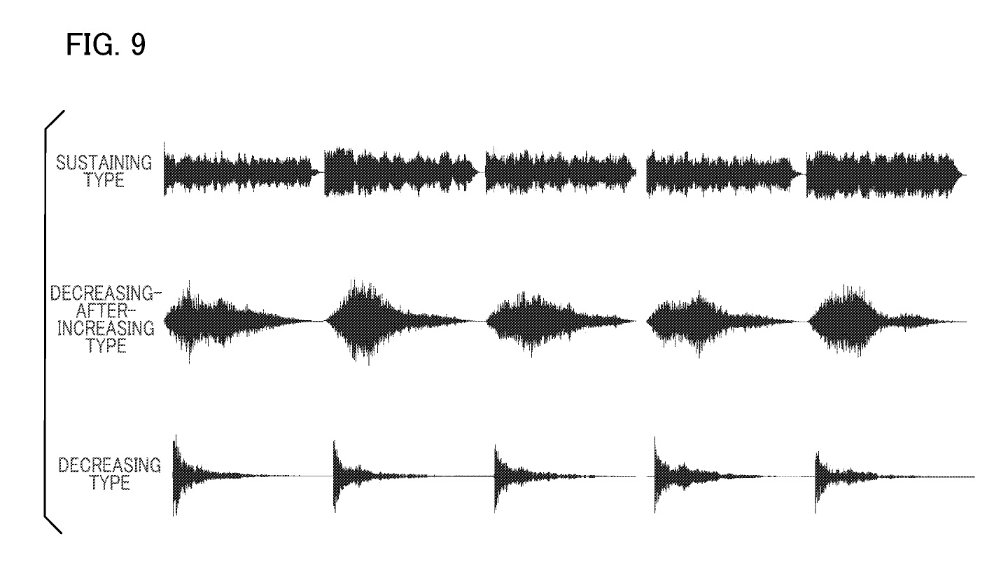

FIG. 9 shows example waveforms of the sound signals V generated by the sound signal generator 245 based on the sound information D. As the figure shows, with regard to the sequentially generated plural sound signals V, a change was cyclically (or more specifically, in a cycle based on the biological cycle) made from the sound signal V corresponding to the first sound information D to the sound signal V corresponding to the second sound information D. FIG. 9 shows that the types of waveforms of the sound signals V are the sustaining type, the decreasing-after-increasing type and the decreasing type. The decreasing type and the decreasing-after-increasing types have already been described with reference to FIGS. 5 and 6. The sustaining type has an amplitude that is generally constant. It is of note that the waveform of the sound linked to heartbeat and that of the ambient sound are not of the sustaining type.

As shown in FIG. 8, the No. 1 sound is silence. In other words, when the No. 1 sound is used, the subject users do not hear anything. The No. 2 sound includes the sound linked to breathing and the ambient sound and it is obtained by causing the sound signal generator 245 to generate the sound signal V based on the sound information BD for a breathing-based cycle and the sound information AD for an ambient sound. Here, the sound information BD for a breathing-based cycle is obtained by generating a chord using a synthesizer. The waveform of the sound signals V generated by the sound signal generator 40 based on selected pieces of the sound information BD are of the sustaining type shown in FIG. 9. The No. 3 sound also includes the sound linked to breathing and the ambient sound, and the only element that differentiates the No. 3 sound from the No. 2 sound is the change in the amplitude of the waveform of the sound signal V generated by the sound signal generator 245 based on the sound information BD for a breathing-based cycle. In other words, with regard to the No. 3 sound, each waveform corresponding to the sound information BD for a breathing-based cycle is of the decreasing-after-increasing type shown in FIG. 9.

The No. 4 sound is a sound inspired by the tones of bells used in Tibetan Buddhism. It includes the sound linked to heartbeat and the sound linked to breathing. The No. 4 sound is obtained by causing the sound signal generator 40 to generate the sound signal V based on the sound information BD for a breathing-based cycle and the sound information HD for a heartbeat-based cycle. Here, the sound information BD for a breathing-based cycle is obtained by sampling bell sounds. The waveforms of the sound signals generated by the sound signal generator 245 based on pieces of the sound information BD are of the decreasing type that FIG. 9 shows. The No. 5 sound includes the sounds linked to heartbeat made by Japanese percussion instruments and also the ambient sound. The sound is obtained by causing the sound signal generator 40 to generate the sound signal V based on the sound information HD for a heartbeat-based cycle and the sound information AD for an ambient sound. Here, the sound information HD for a heartbeat-based cycle is obtained by sampling the sounds of Japanese percussion instruments. The No. 6 sound includes the sound linked to heartbeat and the sound linked to breathing, both using the sound of waves, and the ambient sound. The different sounds, except for the No. 2 and No. 3 sounds, include completely different tones and impart different impressions to the subject users.

In the experiments, the difference between the sleep latency time for the 5 sounds (No. 2 to No. 6), when compared to the sleep latency time for silence (No. 1), was observed. Also observed was the relationship between the sleep latency time and each of the following collateral conditions: the results of the questionnaire answered when the subject users woke up; the subject users' sensitivity for each sound, the subject users' hearing, the number of times the experiments were conducted, climate conditions, the room the subject users were in, and what day of the week it was.

### 2-2. Results of the Experiments

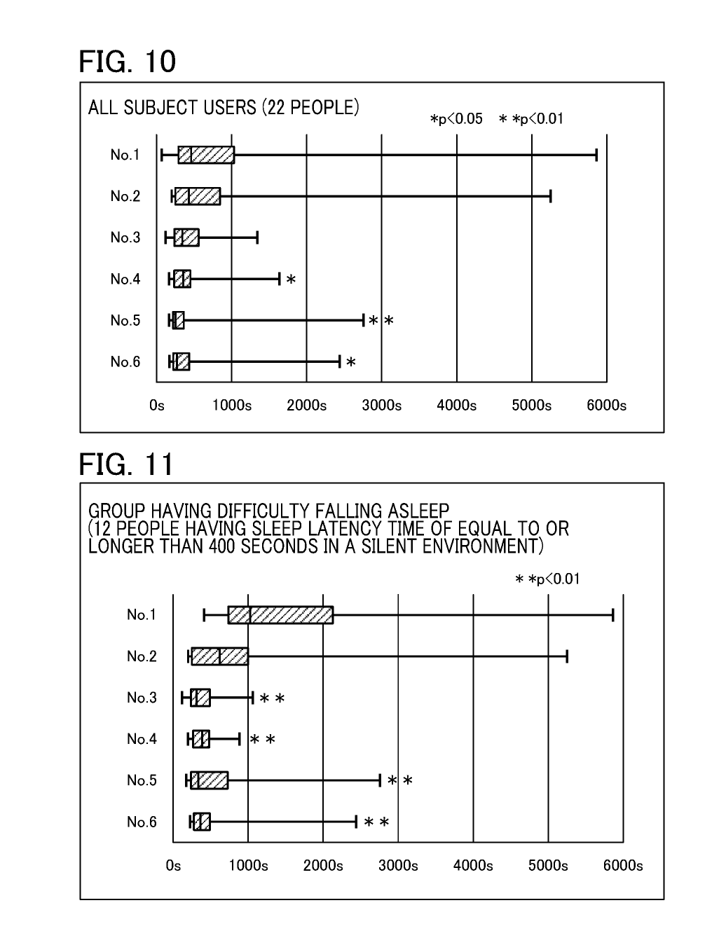

FIG. 10 shows the distribution of the sleep latency time of all 22 subject users for each of the sounds No. 1 through No. 6. Compared to the No. 1 sound (silence), the sounds No. 4, 5 and 6 showed statistically meaningful shortening of sleep latency time. Furthermore, among the 22 subject users, those whose sleep latency time was equal to or longer than 400 seconds in a silent environment were grouped as the group that had difficulty falling asleep. FIG. 11 shows the distribution of the sleep latency time of the group that had difficulty falling asleep (12 people had a sleep latency time equal to or longer than 400 seconds in a silent environment) when they listened to each of the sounds No. 1 through No. 6. The letter “p” in FIGS. 10 and 11 denotes the results of the tests. The single asterisk (*) denotes that there is less than a 5% likelihood for the hypothesis that the sleep latency time does not change from when the subject user is in a silent environment to when the subject user is in an environment with a particular sound. The double asterisks (**) denote that there is less than a 1% likelihood for the hypothesis that the sleep latency time does not change from when the subject user is in a silent environment to when the subject user is in an environment with a particular sound. Furthermore, the horizontal lines in these figures indicate the minimum to maximum sleep latency time, and the shaded portions indicate where measurement results appear highly frequently. The vertical lines in the shaded portions indicate the median. As is apparent from FIG. 10, compared to a silent environment, in an environment in which sounds No. 4, 5 and 6 were present, a statistically meaningful shortening of sleep latency time occurred. Namely it is shown that the subject users fell asleep more quickly when they listened to the sounds compared to when they were in a silent environment. It also indicates that when sounds such as the sounds of bells and drums, as well as synthetic sounds of synthesizers were listened to, the same shortening effect on the sleep latency time occurred. Moreover, it is shown that sound information including such tones is appropriate as the sound information D.

Focusing on the amplitude change in the sound linked to breathing, the No. 2 sound of the sustaining type showed the same results as those obtained in a silent environment. In contrast, the No. 3 sound of the decreasing-after-increasing type showed a shortening effect on sleep latency time. When a change in amplitude was applied to similar tones, different results were obtained. The No. 4 sound is of the decreasing type with the sound decreasing after the point at which the bell rings “gong”. The No. 6 sound is the sound of waves belonging to a combined type of the decreasing-after-increasing type and the decreasing type. Both of these sounds showed a shortening effect on sleep latency time. Focusing on the group having difficulty falling asleep, each of No. 3, 4 and 6 sounds showed noticeable effects as shown in FIG. 11.

Based on the abovementioned experiment results, it can be concluded that the sounds linked to breathing that are of the decreasing-after-increasing type and the decreasing type have a shortening effect on sleep latency time, while sounds of the sustaining type played at a fixed volume do not have such an effect. The breathing cycle BRm is a cycle with a duration of at least around 4 seconds, which is sufficiently long for the subject user to perceive the change in volume. The subject user can readily perceive his/her biological cycle because in the decreasing-after-increasing type and the decreasing type, the volume constantly changes. However, the sustaining type has a fixed volume, and there is no clue, other than the breaks in the cycle, that would assist the subject user in perceiving his/her biological cycle.

### 3. Second Embodiment

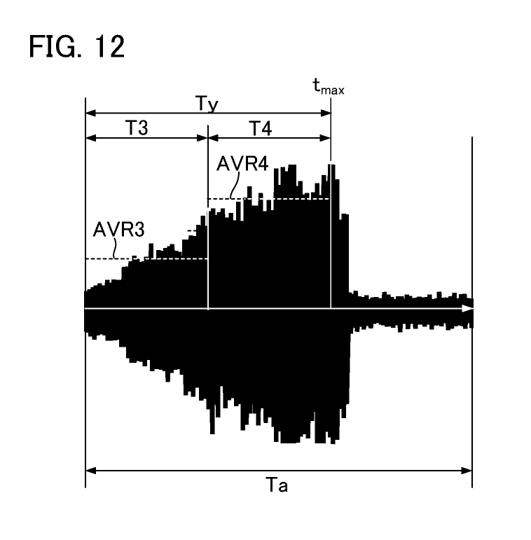

The system 1 of the second embodiments is configured in substantially the same way as the system 1 of the first embodiment, except for the sound information D stored in the storage unit 250. In the second embodiment, in addition to the sound information D described relative to the first embodiment, there is included as the sound information BD for a breathing-based cycle, sound information with the waveform of the sound signal V generated by the sound signal generator 245 as shown in FIG. 12.

In the waveform in this figure, the amplitude first generally increases, and after being maximized the amplitude then sharply decreases. Hereunder, such a waveform is referred to as the “increasing type”. The term “generally increase” refers to a waveform that when viewed over a short time span shows both increases and decreases in amplitude, but when the same waveform is viewed in its entirety it is apparent that there is an overall increase in the amplitude. Regarding the waveform of the sound signal V corresponding to the sound information BD for a breathing-based cycle, the mean amplitude AVR4 of the waveform in the fourth period T4 is larger than the mean amplitude AVR3 of the waveform in the third period T3, when the period Ty, which is the period between the start of the waveform and the maximum amplitude point tₘₐₓ, is divided in half i.e. into the third period T3 that comes first and the fourth period T4 that comes next. Here, the maximum amplitude point tₘₐₓ indicates the time at which the amplitude maximizes in a waveform of the sound information BD for a breathing-based cycle. In an increasing type waveform, the amplitude increases from the start of the waveform until it maximizes. Therefore, just as in the case of the waveform of the decreasing type as shown in FIG. 5, the increasing type waveform allows the volume of the sound to substantially change as compared to the sustaining type, within a single cycle corresponding to the biological cycle. Therefore, by selecting the increasing type of sound information as the sound information BD, the subject user E can more distinctly perceive the cycle based on his/her acquired biological cycle, thus inducing sleep in him/her more quickly after going to bed.

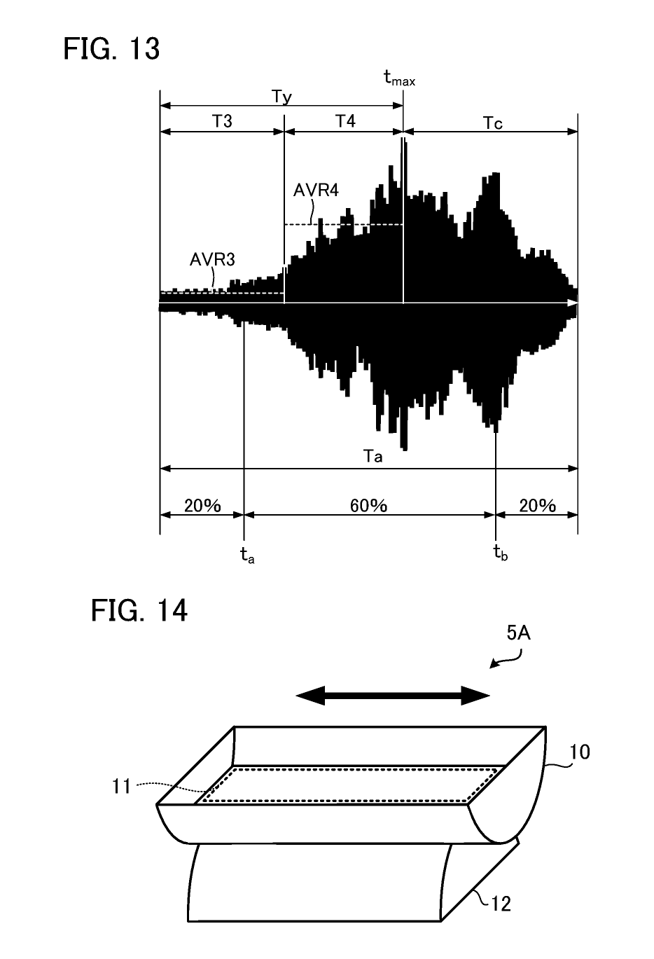

The sound information shown in FIG. 13 may be selected as the sound information BD for a breathing-based cycle. In the waveform shown in this figure, the amplitude generally increases until it is maximized, and after that, it gradually decreases. Such a waveform is one kind of the decreasing-after-increasing type. In this decreasing-after-increasing type waveform, a period Tc exists in which the amplitude gradually decreases after the amplitude is maximized. Regarding also the decreasing-after-increasing type waveform shown in the figure, the mean amplitude AVR4 of the waveform in the fourth period T4 is larger than the mean amplitude AVR3 of the waveform in the third period T3, when the period Ty, which is the period between the start of the waveform and the maximum amplitude point tₘₐₓ, is divided in half, i.e. into the third period T3 that comes first and the fourth period T4 that comes next. Here, the maximum amplitude point tₘₐₓ indicates the time at which the amplitude maximizes in the waveform. Therefore, by selecting a decreasing-after-increasing type of sound information shown in FIG. 13 as the sound information BD, the subject user E can more distinctly perceive the cycle based on his/her acquired biological cycle, thus inducing in him/her sleep more quickly after going to bed. This effect is the same as the effect obtained by the increasing type sound information shown in FIG. 12.

Here, in the increasing type waveform of FIG. 12 and the decreasing-after-increasing type waveform of FIG. 13, it is preferable that the mean amplitude AVR3 be equal to or less than 70% of the mean amplitude AVR4. When AVR3 is equal to or less than 70% of AVR4 the subject user E can more readily perceive his/her own biological cycles. In the examples shown in FIGS. 12 and 13, the time period Ty between the start of the waveform and the maximum amplitude point tₘₐₓ was divided in half. However, it is also possible to divide the period Ty by three or four. In such a case, the mean amplitude of each period generally increases from the start of the waveform at the head of each period towards the maximum amplitude point.

Furthermore, as with the decreasing-after-increasing type of sound information D described in the first embodiment, in a waveform of the sound signal V corresponding to the sound information BD of the second embodiment, when the entire time from the start to the end of the waveform is deemed 100%, it is preferable that the maximum amplitude point tₘₐₓ comes within the range between a time point tₐ, at which 20% of time has passed from the start of the waveform, and a time point t_{b}, at which 20% of time remains until the end of the waveform. If the maximum amplitude point tₘₐₓ is within this range, the subject user E can more distinctly perceive the process in which the amplitude increases to its maximum point and the process in which the amplitude decreases from the maximum point. Accordingly, it is expected that the subject user E will be able to more readily perceive the volume change in the cycle based on his/her acquired biological information, and thus be induced to fall asleep. It is noted that, preferably, all pieces of sound information D stored in the storage unit 250 should be of either the aforementioned increasing or decreasing-after-increasing type. However, not all pieces of sound information D stored in the storage unit 250 and from which the sound signal generator 245 generates sound signals need be of the abovementioned increasing type or the decreasing-after-increasing type, and only at least one of the pieces of sound information D stored in the storage unit 250 needs to be of such a type. In other words, it is sufficient for the amplitude of the waveform of the sound signal V corresponding to at least one piece of sound information D that is generated by the sound information generator 245 to generally increase from the start of the waveform towards the maximum amplitude point tₘₐₓ.

### 4. Third Embodiment

In each of the above-mentioned first and the second embodiments, the sound information D is changed in a cycle based on the biological cycle. In contrast, in the third embodiment, sound information is not changed but is repeated. A repeat cycle here is a cycle based on the biological cycle, as the change cycle in the above-mentioned first and the second embodiments. A sound signal generation device of the third embodiment repeatedly generates a sound signal based on the same sound information in a cycle corresponding to a breathing cycle. Furthermore, in the above-mentioned first and second embodiments, there are stored in the storage unit 250 plural pieces of sound information D including plural pieces of sound information BD for a breathing-based cycle, plural pieces of sound information HD for a heartbeat-based cycle, and plural pieces of sound information AD for an ambient sound. In the first embodiment, at least one piece of the sound information D is a decreasing type (FIG. 5) or decreasing-after-increasing type (FIG. 6), and in the second embodiment, at least one piece of the sound information D is an increasing type (FIG. 12) or decreasing-after-increasing type (FIG. 13). In the third embodiment, however, the storage unit 250 stores plural pieces of sound information BD for a breathing-based cycle only, and each one of these plural pieces of sound information BD is a decreasing type, increasing type, or decreasing-after-increasing type. The third embodiment is substantially the same as the first embodiment except for the above differences, and in the following description, the same reference numerals as those of the first embodiments are assigned to the same parts as those in the first embodiment, and description of these parts is omitted as appropriate.

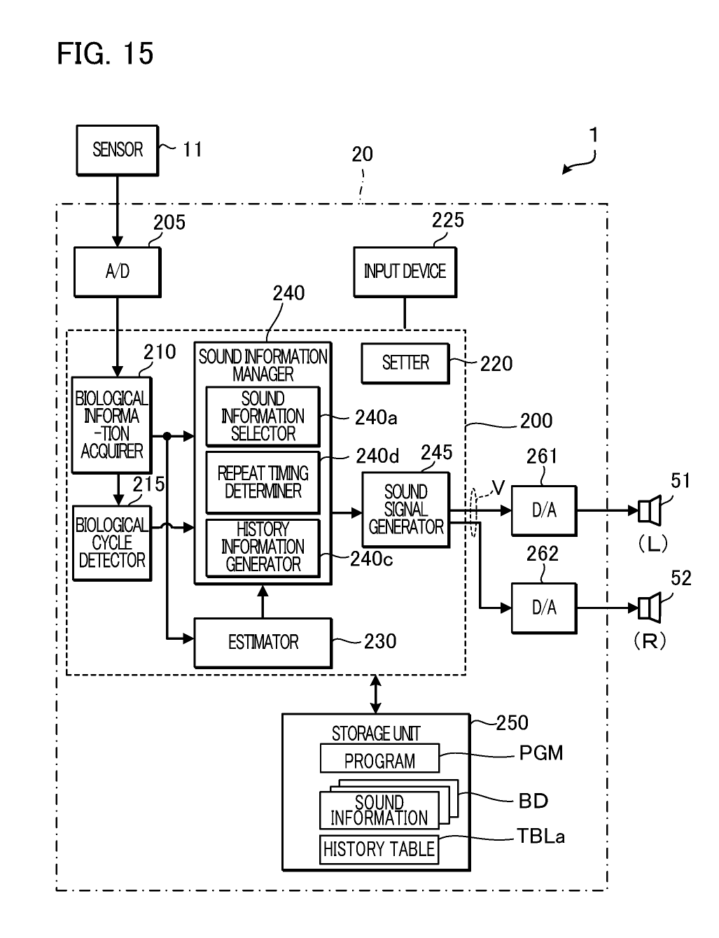

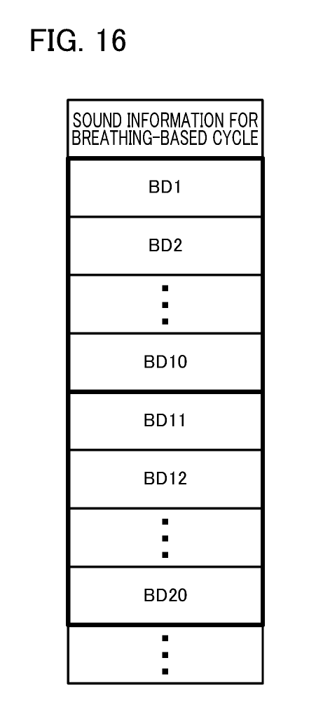

FIG. 15 is a block diagram showing a functional configuration of a sound signal generation device 20 of the present embodiment. A system 1 of FIG. 15 is the same as that of the first embodiment except that it includes a repeat timing determiner 240d instead of the change timing determiner 240b. The repeat timing determiner 240d determines a repeat timing so that the sound information BD is repeatedly generated in a cycle based on biological information obtained by the biological information acquirer 210 (more specifically, in a cycle based on the breathing cycle BRm detected by the biological cycle detector 215 based on the biological information obtained by the biological information acquirer 210). As shown in FIG. 16, there are stored in the storage unit 250 plural pieces of sound information BD (BD1, BD2 . . . ) for a breathing-based cycle. As described above, each of these pieces of sound information BD for a breathing-based cycle is one of a decreasing type, increasing type, or decreasing-after-increasing type, and the sound information selector 240a selects at random any one of pieces of sound information BD included in a group from which the sound information BD for a breathing-based cycle is to be selected. It is of note that since the present embodiment uses the sound information BD for a breathing-based cycle only based on which a sound signal is generated, the first, second, and third sound signal generators 410, 420 and 430 or mixers 451 and 452 as in the first embodiment are not necessarily provided.

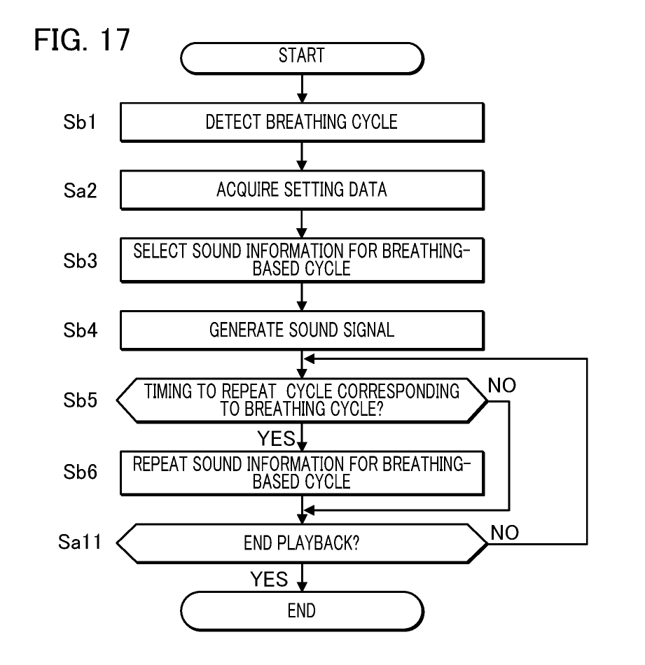

In the above configuration, the sound signal generation device 20 of the present embodiments operates as follows. FIG. 17 shows an example flow of operations performed by the sound signal generation device 20.

The biological cycle detector 215 first detects the breathing cycle BRm of the subject user E based on the detection signals indicating the biological information of the subject user E acquired by the biological information acquirer 210 (Sb1). The sound information selector 240a then acquires from the storage unit 250 the setting data that has been set by the setter 220 (Sa2) and determines, based on the setting data, the group from which the sound information BD for a breathing-based cycle is selected. The sound information selector 240a selects, according to a prescribed rule (at random, in the present example), a piece of the sound information BD for a breathing-based cycle (Sb3), the sound signal generator 245 then generating the sound signal V based on the sound information BD after reading from the storage unit 250 the selected piece of sound information BD for a breathing-based cycle (Sb4). As will be understood from the following description, the selected sound information BD is repeatedly used to generate the sound signal V. Since each of the pieces of sound information BD stored in the storage unit 250 is one of a decreasing type, increasing type, or decreasing-after-increasing type, the subject user E is able to perceive his/her biological cycles even if the same sound information BD is repeatedly played.

Subsequently, the repeat timing determiner 240d determines whether or not the current time is the repeat timing based on a cycle corresponding to the breathing cycle BRm of the subject user E (Sb5). When the determination conditions in step Sb5 are met, the repeat timing determiner 240d supplies to the sound signal generator 245 a timing signal that instructs the sound signal generator 245 to repeatedly generate a sound signal based on the sound information BD that is currently being generated. Once the timing signal has been supplied, the sound signal generator 245 generates the sound signal V based on the sound information BD that is currently being generated (Sb6).

Thus, the present embodiment also is expected to lead the subject user E into a relaxed state, since the subject user E is able to perceive his/her biological cycles with the repeatedly played sound information BD being one of a decreasing type, increasing type, or decreasing-after-increasing type, and the sound information BD is repeatedly played in a cycle based on his/her biological information. Accordingly, a person will feel relaxed and thus be able to fall asleep quickly.

In the present embodiment, the pieces of sound information BD only are stored in the storage unit 250 and one selected from the stored pieces of sound information BD is repeatedly played in a cycle based on the breathing cycle BRm. As an alternative, plural pieces of sound information HD for a heartbeat-based cycle (each being one of a decreasing type, increasing type, or decreasing-after-increasing type) only may be stored. In this case, one selected from the stored pieces of sound information HD is repeatedly played in a cycle based on the heartbeat cycle HRm. Alternatively, both the pieces of sound information BD for a breathing-based cycle and the pieces of sound information HD for a heartbeat-based cycle may be stored in the storage unit 250, such that one selected from the stored pieces of sound information BD for a breathing-based cycle is repeatedly played in a cycle based on the breathing cycle BRm and one selected from the stored pieces of sound information HD for a heartbeat-based cycle is repeatedly played in a cycle based on the heartbeat cycle HRm. By doing so, the same effects as those of the third embodiment can be achieved.

### 5. Modifications

The present invention is not limited to the above-mentioned embodiments and can be applied and modified variously, for example as described below. Further, any of the following applications and modifications can be selected for use, or the following applications and modifications can be combined as appropriate.

### Modification 1

In each of the embodiments described above, the sensor 11 of a sheet-form is used to detect the biological information of the subject user E, but the present invention is not limited to a sheet-form sensor, and any kind of sensor may be used as long as it detects biological information. For example, electrodes of a first sensor may be attached to the forehead of the subject user E so as to detect the brain waves (α (alpha) wave, β (beta) wave, δ (delta) wave, θ (theta) wave, etc.) of the subject user E. A second sensor may additionally to or alternatively to the first sensor be worn on the left wrist of the subject user E to detect, for example, a change in pressure of the radial artery which is the pulse wave. The pulse wave is synchronized with a heartbeat, and hence the second sensor detects the heartbeat indirectly. Furthermore, a third sensor for detecting acceleration may be additionally to or alternatively to at least one of the first sensor and the second sensor provided between the head of the subject user E and a pillow, the third sensor detecting breathing, heartbeat, etc., based on the body motion of the subject user E. As other sensors for detecting biological information, any one of pressure sensors, pneumatic sensors, vibration sensors, optical sensors, ultrasonic Doppler sensors, RF Doppler sensors, laser Doppler sensors, etc., may be used. In a case in which the biological cycle detector 215 detects brain waves, when the estimator 230 estimates the physical and mental state of the subject user E, a resting state with relatively little body motion and in which β (beta) waves are dominant in the brain wave patterns of the subject user E is estimated by the estimator 230 as “awake”. A state in which θ (theta) waves appear in the brain wave patterns of the subject user E is estimated by the estimator 230 as “light sleep”. A state in which δ (delta) waves appear in the brain wave patterns of the subject user E is estimated by the estimator 230 as “deep sleep”. A state in which breathing is shallow and irregular although θ (theta) waves appear in the brain wave patterns of the subject user E is estimated by the estimator 230 as “REM sleep”. To perform this estimation, various other procedures known in the art may be used.

### Modification 2

In the abovementioned embodiments, plural pieces of sound information BD for a breathing-based cycle are managed in plural groups, plural pieces of sound information HD for a heartbeat-based cycle are managed in plural groups, and plural pieces of sound information AD for an ambient sound are managed in plural groups. For this reason, the sound information selector 240a randomly selects a single piece of sound information BD for a breathing-based cycle from one part (i.e., from one group) among the plural pieces of sound information BD for a breathing-based cycle that are stored in the storage unit 250. The sound signal generator 245 then generates the sound signal V based on the selected piece of sound information BD for a breathing-based cycle in a cycle based on the breathing cycle BRm. The present invention is not limited to the above, and all pieces of sound information BD for a breathing-based cycle that are stored in the storage unit 250 may be the object of selection. Similarly, the sound information selector 240a may randomly select a single piece of sound information HD for a heartbeat-based cycle from one part (i.e., from one group) among the plural pieces of sound information HD for a heartbeat-based cycle that are stored in the storage unit 250. The sound signal generator 245 then generates, in a cycle based on the heartbeat cycle HRm, the sound signal V based on the selected sound information HD for a heartbeat-based cycle. The present invention is not limited to the above, and all pieces of sound information HD for a heartbeat-based cycle that are stored in the storage unit 250 may be the object of selection. Moreover, the groups from which the sound information D (the second sound information D) is selected may be changed as appropriate according to a prescribed rule.

### Modification 3

In the above-described first and second embodiments, the sound information AD for an ambient sound is changed to a new sound information AD in a predetermined cycle. However, the present invention is not limited thereto, and the sound information AD for an ambient sound need not necessarily be changed from the first to second sound information AD, but may remain the same.

### Modification 4

In each of the embodiments described above, the history information generator 240c stored the following in the history table TBLa in association with the processing time: physical and mental state estimated by the estimator 230; and the identifier(s) of the selected piece(s) of sound information D (at least one piece of information from the sound information BD for a breathing-based cycle, the sound information HD for a heartbeat-based cycle, or the sound information AD for an ambient sound). Therefore, by referring to the history table TBLa, the kinds of sound information D preferable for the subject user E, for example those resulting in a shortened time in which to fall asleep after going to bed, may be specified. In such a case, one of or combinations of two or more of the following may be specified from the identifiers of the sound information stored in the history table TBLa: the group of pieces of sound information BD for a breathing-based cycle; the group of pieces of sound information HD for a heartbeat-based cycle; and the group of pieces of sound information AD for an ambient sound. Specifically, it is possible to specify a combination of groups that is appropriate for transition states such as from “awake” to “light sleep” and from “light sleep” to “deep sleep”. Thus, by referring to the history table TBLa, the sound information selector 240a may automatically make a change according to the estimated physical and mental state for at least one of the following: the group from which the sound information BD for a breathing-based cycle is selected; the group from which the sound information HD for a heartbeat-based cycle is selected; and the group for which the sound information AD for an ambient sound is selected.

Moreover, when the subject user E has difficulty falling asleep, i.e., if he/she takes more time than average to fall asleep after going to bed, the sound information selector 240a may refer to the history table TBLa and automatically change to a group that has a higher possibility of more quickly inducing sleep in him/her. In this way, a quality of sleep may be greatly improved by reflecting the assessment of the subject user E's state of sleep (specifically, the estimated physical and mental state) in the selection of the sound information D.

### Modification 5

In each of the embodiments described above, sound information is indicated as an example of contents that lead the subject user E into sound sleep. However, the present invention is not limited to sound information and other stimulants such as light and vibration, or alternatively with addition of other stimulants, may be used to improve the quality of sleep of the subject user E. For example, the present invention may be adapted for use with a rocking bed 5A as shown in FIG. 14. The rocking bed 5A is configured to serve as a baby bed for infants, having a main bed unit 10 set on top of a base unit 12. The main bed unit 10 rocks from left to right (as viewed from the perspective shown in FIG. 14) above the base unit 12 so as to induce sound sleep in infants.

Inside the base unit 12 of the rocking bed 5A, a motor is attached to rock the main bed unit 10. In the storage unit of the rocking bed 5A, plural pieces of driving information for driving the motor are stored. These pieces of driving information are waveform data for driving the motor. A driving controller of the rocking bed 5A drives the motor using driving signals obtained by DA converting the waveform data read from the storage unit. In doing so, a biological cycle detector of the rocking bed 5A detects the infant's biological cycles based on the biological information output from the sensor, and changes from first driving information to second driving information in a cycle based on the biological cycle so as to rock the main bed unit 10. Here, it is preferable that the subject user, namely the infant, is able to perceive the cycle based on his/her biological cycles in order to induce sound sleep in him/her. For this reason, at least one piece of driving information stored in the storage unit is preferably selected from the above-described increasing, decreasing, or decreasing-after-increasing types. In other words, within a cycle based on biological cycle, the bed is rocked multiple times, with the amplitude of the multiple rocking motions being changed in a similar manner to the waveforms of the above-described increasing, decreasing, or decreasing-after-increasing types. By changing from the first driving information to the second driving information in a biological cycle, the rocking of the bed changes similarly to the waveforms of the decreasing-after-increasing and decreasing types shown in FIG. 9.

### Modification 6

In each of the above-described embodiments, the sound signal generator 245 acquires the sound information D from the storage unit 250. However, the present invention is not limited thereto, and as long as the sound information D can be acquired, the sound information D may be stored anywhere. For example, the sound signal generation device 20 may have a communication unit that can communicate with a server connected to a communication network, with the sound information D stored in the server being acquired via the communication unit. In this case, the server may be located within the same facility as the sound generation device 20, or may be located outside the facility. In other words, the sound signal generator 245 may acquire the sound information D via a communication network such as the Internet.

### DESCRIPTION OF REFERENCE SIGNS

1 . . . system, 11 . . . sensor, 20 . . . sound signal generation device, 51 and 52 . . . speakers, 200 . . . controller, 210 . . . biological information acquirer, 215 . . . biological cycle detector, 220 . . . setter, 225 . . . input device, 230 . . . estimator, 240 . . . sound information manager, 240a . . . sound information selector, 240b . . . change timing determiner, 240c . . . history information generator, 245 . . . sound signal generator, 250 . . . storage unit, D (AD, BD, HD) . . . sound information, V (V_{AD}, V_{BD}, V_{HD}) . . . sound signal, PGM . . . program, TBLa . . . history table, T1 . . . first period, T2 . . . second period, T3 . . . third period, T4 . . . fourth period

What is claimed is:

1. A sound signal generation device comprising:
a biological information acquirer configured to acquire biological information of a subject user;
a change timing determiner configured to determine a change timing that allows a first piece of sound information to be changed to a second piece of sound information in a cycle corresponding to the biological information acquired by the biological information acquirer; and
a sound signal generator configured to generate a sound signal based on the second piece of sound information at a timing determined by the change timing determiner,
wherein an amplitude of a waveform of a sound signal generated by the sound signal generator based on at least one piece of sound information, among a plurality of pieces of sound information including the first piece of sound information and the second piece of sound information, generally decreases from a maximum amplitude point, in which the amplitude is maximized, towards the end of the waveform, or generally increases from the start of the waveform towards a maximum amplitude point, in which the amplitude is maximized.

2. The sound signal generation device according to claim 1,
wherein, when a period between the maximum amplitude point and the end of the waveform of the at least one piece of sound information is divided in half into a first period and a second period, the second period coming after the first period, a mean amplitude of the waveform in the second period is smaller than a mean amplitude of the waveform in the first period.

3. The sound signal generation device according to claim 1,
wherein, when a period between the start of the waveform and the maximum amplitude point of the at least one piece of sound information is divided in half into a third period, and a fourth period, the fourth period coming after the third period, a mean amplitude of the waveform in the fourth period is larger than a mean amplitude of the waveform in the third period.

4. The sound signal generation device according to claim 1,
wherein, in the at least one piece of sound information, when a time from the start to the end of the waveform is deemed 100%, the maximum amplitude point comes within the range between a time point at which 20% of time has passed from the start of the waveform, and another time point at which 20% of time remains until the end of the waveform.

5. A sound signal generation device comprising:
a biological information acquirer configured to acquire biological information of a subject user;
a repeat timing determiner configured to determine a repeat timing that allows a piece of sound information to be repeatedly generated in a cycle corresponding to the biological information acquired by the biological information acquirer; and
a sound signal generator configured to generate a sound signal based on the piece of sound information at a timing determined by the repeat timing determiner,
wherein an amplitude of a waveform of a sound signal generated by the sound signal generator based on the piece of sound information generally decreases from a maximum amplitude point, in which the amplitude is maximized, towards the end of the waveform, or generally increases from the start of the waveform towards a maximum amplitude point, in which the amplitude is maximized.

6. The sound signal generation device according to claim 5,
wherein, when a period between the maximum amplitude point and the end of the waveform of the piece of sound information is divided in half into a first period and a second period, the second period coming after the first period, a mean amplitude of the waveform in the second period is smaller than a mean amplitude of the waveform in the first period.

7. The sound signal generation device according to claim 5,
wherein, when a period between the start of the waveform and the maximum amplitude point of the piece of sound information is divided in half into a third period and a fourth period, the fourth period coming after the third period, a mean amplitude of the waveform in the fourth period is larger than a mean amplitude of the waveform in the third period.

8. The sound signal generation device according to claim 5,
wherein, in the piece of sound information, when a time from the start to the end of the waveform is deemed 100%, the maximum amplitude point comes within the range between a time point at which 20% of time has passed from the start of the waveform, and another time point at which 20% of time remains until the end of the waveform.

9. A sound signal generation method comprising:
acquiring biological information of a subject user;
determining a change timing that allows a first piece of sound information to be changed to a second piece of sound information in a cycle corresponding to the biological information; and
generating a sound signal based on the second sound piece of information at the determined timing,
wherein an amplitude of a waveform of a sound signal generated based on at least one piece of sound information, among a plurality of pieces of sound information including the first piece of sound information and the second piece of sound information, generally decreases from a maximum amplitude point, in which the amplitude is maximized, towards the end of the waveform, or generally increases from the start of the waveform towards a maximum amplitude point, in which the amplitude is maximized.

10. The sound signal generation method according to claim 9,
wherein, when a period between the maximum amplitude point and the end of the waveform of the at least one piece of sound information is divided in half into a first period and a second period, the second period coming after the first period, a mean amplitude of the waveform in the second period is smaller than a mean amplitude of the waveform in the first period.

11. The sound signal generation method according to claim 9,
wherein, when a period between the start of the waveform and the maximum amplitude point of the at least one piece of sound information is divided in half into a third period and a fourth period, the fourth period coming after the third period, a mean amplitude of the waveform in the fourth period is larger than a mean amplitude of the waveform in the third period.

12. The sound signal generation method according to claim 9,
wherein, in the at least one piece of sound information, when a time from the start to the end of the waveform is deemed 100%, the maximum amplitude point comes within the range between a time point at which 20% of time has passed from the start of the waveform, and another time point at which 20% of time remains until the end of the waveform.

13. A sound signal generation method comprising:
acquiring biological information of a subject user;
determining a repeat timing that allows a piece of sound information to be repeatedly generated in a cycle corresponding to the acquired biological information; and
generating a sound signal based on the piece of sound information at the determined timing,
wherein an amplitude of a waveform of a sound signal generated based on the piece of sound information generally decreases from a maximum amplitude point, in which the amplitude is maximized, towards the end of the waveform, or generally increases from the start of the waveform towards a maximum amplitude point, in which the amplitude is maximized.

14. The sound signal generation method according to claim 13,
wherein, when a period between the maximum amplitude point and the end of the waveform of the piece of sound information is divided in half into a first period and a second period, the second period coming after the first period, a mean amplitude of the waveform in the second period is smaller than a mean amplitude of the waveform in the first period.

15. The sound signal generation method according to claim 13,
wherein, when a period between the start of the waveform and the maximum amplitude point of the piece of sound information is divided in half into a third period and a fourth period, the fourth period coming after the third period, a mean amplitude of the waveform in the fourth period is larger than a mean amplitude of the waveform in the third period.

16. The sound signal generation method according to claim 13,
wherein, in the piece of sound information, when a time from the start to the end of the waveform is deemed 100%, the maximum amplitude point comes within the range between a time point at which 20% of time has passed from the start of the waveform, and another time point at which 20% of time remains until the end of the waveform.
